(19) United States
(12) Patent Application Publication
(10) Pub. No.: US 2007/0275373 A1
(43) Pub. Date: Nov. 29, 2007

(54) Method of fingerprinting tissue samples

(76) Inventors: Lois Smith, West Newton, MA (US); Shu-Ching Shih Jaminet, Cambridge, MA (US)

Correspondence Address: David S Resnick;Nixon Peabody, 100 Summer Street, Boston, MA, 02110, US

(21) Appl. No.: 10/512,253

(22) PCT Filed: Apr. 25, 2003
(86) PCT No.: PCT/US03/12964

§ 371 Date: Jan. 4, 2005

Related U.S. Application Data
(60) Provisional application No. 60/375,895, filed on Apr. 26, 2002.

(51) Int. Cl. C12Q 1/68; C12N 15/63

(52) U.S. Cl. 435/6; 435/320.1

(57) ABSTRACT

The present invention is directed to methods of determining a copy number of nucleic acids in a biological specimen. More specifically, the invention provides a method for determining an absolute copy number of a transcript or a plurality of transcripts of interest in a biological specimen the size of which is determined using an external control. The invention further provides a method for diagnosis and prognosis of diseases associated with changes in a transcript copy number by measuring an absolute copy number of the transcript The invention also provides a kit for measuring absolute copy number of a transcript or a plurality or transcripts. The invention further provides a method of creating a quantitative “finger-print” of a disease-related gene expression pattern for screening to identify susceptibility to a disease, and disease diagnostic and prognostic purposes.

This invention was made with Government Support under Contract No. NO1-CN-85166 MAO awarded by the National Cancer Institute. The U.S. Government has certain rights in the invention.

### BACKGROUND OF THE INVENTION

1. Field of the Invention

The present invention relates to methods of determining a copy number of nucleic acids in a biological specimen. The invention further relates to method of creating a quantitative “fingerprint” of a disease-related gene expression pattern for screening to identify susceptibility to a disease, and disease diagnostic and prognostic purposes.

2. Background

Detection and quantification of differentially expressed genes would be useful in diagnosis, prognosis and potentially also in designing treatment alternatives, specifically when pharmacogenomic data are to accumulate in a number of pathological conditions such as different benign and malignant tumors, neurological disorders, heart disease and autoimmune disorders, as well as infectious diseases.

The methods for nucleic acid and specifically mRNA detection and quantification have traditionally included hybridization-based methods such as the Northern-blot hybridization, ribonuclease protection assay, and reverse transcriptase polymerase chain reaction (RT-PCR). However, between different samples these methods are only useful for roughly estimating the amount of each transcript compared to amount of a standard such as a housekeeping gene transcript. Even within each sample, accurate quantification of nucleic acids is complicated or not possible at all using the traditional techniques.

The different RT-PCR based techniques are the most suitable quantification method for diagnostic purposes, because they are very sensitive and thus require only a small sample size which is desirable for a diagnostic test.

Absolute quantification of mRNA copy numbers in a sample is required to compare transcripts between samples and within the same sample. Determining the copy number of a nucleic acid in a biological sample using PCR based methods is complicated because of the inherent non-linear nature of the PCR reaction. Amplification of the cDNA template is linear only under limited conditions, which depend upon the amount of template, primers and polymerase enzyme used in the reaction mixture, as well as the size and sequence of the primers and the target sequence, and the number of cycles during the amplification. Often, the linear phase of the PCR must be determined separately which may involve sampling of the PCR reactions at different time points or performing the PCR using different dilutions of the template. Further, because of differences in the amplification efficiency between different template nucleic acid sequences, the starting quantities of different PCR products cannot be compared directly even in the linear range. Detection of PCR products has traditionally been performed after amplification is completed. Typically, an aliquot of the PCR reaction product is size separated by agarose gel electrophoresis, stained with ethidium bromide, and visualized with ultraviolet light. Alternatively, the primers may be labeled with a fluorescent dye or a radioactive molecule. Comparison of band intensities between samples allows one to qualitatively estimate the relative starting concentrations of templates amplified, but this method is not quantitative and does not result in determination of the absolute copy number.

A number of quantitative RT-PCR based methods have been described including RNA quantification using PCR and complementary DNA (cDNA) arrays (Shalon et al., Genome Research 6(7):639-45, 1996; Bernard et al., Nucleic Acids Research 24(8): 1435-42, 1996), solid-phase mini-sequencing technique (U.S. Pat. No. 6,013,431, Suomalainen et al. Mol. Biotechnol. June;15(2):123-31, 2000), ion-pair high-performance liquid chromatography (Doris et al. J. Chromatogr. A May 8;806(1):47-60, 1998), and 5′ nuclease assay or real-time RT-PCR (Holland et al. Proc Natl Acad Sci USA 88: 7276-7280, 1991).

However, accurate comparison of the changes in the amount of a number of transcripts during a pathological condition, requires obtaining an absolute copy number of mRNA transcripts per cell. Therefore, the methods that use only one control, internal or external, to allow quantification of the specific sequence(s) in the reaction mixture compared to another sequence in the same reaction mixture, are insufficient. Also, a convenient diagnostic assay should be amenable to automation and the equipment should be easy to use and relatively inexpensive.

For example, RNA quantification on a cDNA array after PCR can measure the intensity of a signal from a number of samples for each gene message if a standard amount of sample is applied. However, to compare different genes within the same sample or across samples one must obtain both accurate copy number of the transcripts and accurate knowledge of the amount of sample applied. To compare, for example, variably sized tissue biopsy samples from an individual affected with a progressive disease, would require that the weight of the sample or the number of the cells in the sample be determined. Only after knowing how much material was used to give a certain copy number of transcripts can the copy number be used to accurately compare the gene expression pattern in different samples.

Solid-phase mini-sequencing method suffers from the same drawback. The method uses a homologous sequence which is differentiated using a bi-allelic polymorphic sequence present, for example in a plasmid, as an internal, competitive PCR control. The transcript level is measured against the internal control the amount of which is predetermined. However, to determine the absolute copy number, a predetermined copy number of a plasmid template for each transcript that needs to be measured has to be mixed into each sample limiting the number of transcripts that can be measured from one sample at the same time.

The ion-pair high-performance liquid chromatography is a highly accurate method for determining the copy number of any transcript in a sample. Like solid-phase mini-sequencing, this method relies upon a competitive RT-PCR and a subsequent analysis of the PCR products using ion-pair high-performance liquid chromatography. The major problem using this method is the sophisticated equipment which is not available in a general diagnostic laboratory.

The development of 5′ nuclease assays represents an advance in nucleic acid quantification. This approach utilizes the 5′-3′ exonuclease activity of Thermus aquaticus (Taq) polymerase and either a dual labeled probe annealed to a target sequence where the release of a fluorogenic tag from the 5′ end of the probe is proportional to the PCR product or uses a SYBR Green I dye to detect and verify PCR product by melting curve analysis. This method is applicable for validation of samples containing cDNA fragments prepared with gene-specific primers. SYBR Green I dye intercalation into the minor groove of double-stranded DNA reaches an emission maximum at 530 nm and the emission can be measured in ‘real time’, where the increase in emission intensity is recorded a per-cycle basis. This method allows an easy relative quantification of PCR products.

Thus, it would be useful to develop a method which allows absolute mRNA transcript quantification in the presence of precisely quantified cDNA template and without the current far too labor intensive methods of weighing tissue samples or counting the cells. Such a method would allow comparison not only of one transcript in different tissue samples but also comparison of several transcripts within one sample and between several different samples. Such a method would be useful for disease screening, and diagnostic and prognostic purposes.

### SUMMARY OF THE INVENTION

Therefore it is the purpose of the present invention to provide a method for absolute quantification of gene transcripts. More specifically, the invention provides a method for determining an absolute copy number of a transcript or a plurality of transcripts of interest in a biological specimen the size of which is determined using an external control. The invention further provides a method for diagnosis and prognosis of diseases associated with changes in a transcript copy number by measuring an absolute copy number of the transcript. The invention also provides a kit for measuring absolute copy number of a transcript or a plurality of transcripts.

In one embodiment, the invention provides a method of determining an absolute copy number of a transcript. The method comprises the steps of preparing a “first standard set” comprising a serial dilution of at least two different dilutions of a first vector in a buffer, the vector comprising a vector backbone and a sufficient portion of the coding region of a housekeeping gene. The first vector DNA is isolated, and the number of copies of the first vector DNA is determined. The isolated first vector DNA is diluted so that the number of the first vector DNA copies in each dilution of the serial dilution samples is known. The first standard set may comprise sequences of one or more housekeeping genes in either one vector or in several vectors.

The method further comprises preparing a “second standard set” comprising a serial dilution of at least two different dilutions of a second vector in a buffer, wherein the second vector comprises a vector backbone and a sufficient portion of the coding region of a gene of interest. Like the first vector, the second vector DNA is isolated, and the number of the second vector DNA copies is determined, whereafter the isolated second vector DNA is serially diluted so that the number of the second vector DNA copies in each dilution of the the serial dilution samples is known. The second standard set may comprise sequences of one or more genes of interest.

A PCR reaction is performed using the first standard set as a template with primers selected from a constitutively expressed cDNA (“housekeeping primers”) capable of amplifying the housekeeping gene cloned into the first vector. The amount of PCR product is measured at a linear range of the PCR reaction using each dilution in the serial dilution and plotting the amount of amplified PCR product at the time point against the known copy numbers of the vector in the dilution thereby creating a first standard curve for the housekeeping gene.

A PCR reaction of the second standard set is performed with primers capable of amplifying the gene of interest cDNA (“gene of interest primers” ) cloned into the second vector. Again, the amount of the amplified PCR product is measured at a linear range of the PCR reaction using each dilution as a template and plotting the amount of amplified PCR product at the time point against the known copy number of the vector in the dilution thereby creating a second standard curve for the gene of interest.

To determine an absolute copy number of a gene of interest in a biological sample, a biological specimen is obtained and total RNA is isolated from the biological specimen. Alternatively, a mRNA can be directly isolated from a biological specimen without isolating the total RNA first. Complementary DNA (cDNA) is produced from either the isolated total RNA or isolated mRNA using a reversetranscriptase enzyme. In some applications, isolation is optional, and the RNA reversetranscription and amplification can be performed from a crude biological specimen.

A PCR reaction is performed using the cDNA and using at least two sets of primers. The first set of primers are the housekeeping primers, which may be one pair or plurality of pairs of primers, and the second set of primers are the primers designed to amplify the gene(s) of interest, which may be one pair or plurality of primer pairs. The amount of the PCR product amplified with the housekeeping primers using the RNA from the biological specimen is measured at the same time point as for the first standard and the copy number of the housekeeping gene is determined from the first standard curve. The amount of the PCR product amplified with the gene of interest primers is measured at the same time point as for the second standard and the copy number of the housekeeping gene is determined from the second standard curve. The ratio of copy numbers of housekeeping gene and gene of interest is determined and that shows the copy number of an expressed gene of interest compared to the copy number of the expressed housekeeping gene in the biological specimen.

Once the copy numbers of the housekeeping gene and the gene of interest in the biological specimen are known, i.e. read from the standard curves, the absolute copy number is determined by taking a ratio of the copy number of the gene of interest against the copy number of the housekeeping gene. The housekeeping gene thereby serves a function of an internal “size standard” for each sample.

In one preferred embodiment, only one standard vector is used referred to as “dual standard.” The dual standard vector comprises an amplifiable nucleic acid fragment of at least one housekeeping gene and an amplifiable nucleic acid fragment of at least one gene of interest. Alternatively, the dual standard vector comprises one amplifiable fragment of a housekeeping gene and two or more amplifiable fragments of genes of interest. In embodiments using the dual standard, only one standard dilution set is necessary and a PCR amplification can be performed using both primers amplifying the housekeeping gene(s) and the gene(s) of interest in the same reaction. Alternatively, the vector dilutions can be added to the same PCR amplification tube or well with the nucleic acids from the biological specimen. The only requirement is that the primers for the different genes in the vector can be differentiated. This can be achieved, using different labels for different PCR primers including dyes, such as fluorescent dyes. If the dual standard is amplified in a separate reaction from the sample from the biological specimen, the amplicons representing the different genes may also be differentiated by size.

The invention also provides a kit for determining an absolute copy number of a transcript or a plurality of transcripts comprising a first standard set comprising a serial dilution of a housekeeping gene vector or serial dilutions of a plurality of housekeeping gene vectors and a second standard comprising a serial dilution of a gene of interest vector or serial dilutions of plurality of gene of interest vectors in a RT-PCR compatible buffer, RT-PCR buffer, polymerase enzyme, mixture of nucleotides (dATP, dCTP, dGTP, dTTP), primers for amplifying the first and second standard, and an information sheet comprising instructions of how to determine the absolute copy number of a transcript of interest using the first and second normalizing standards.

In a further embodiment, the invention provides a database comprising tissue fingerprint profiles which have been created using the methods as described above. A “tissue fingerprint” as used herein refers to a set of transcripts with a specific transcription pattern in a particular biological state. For example, a fingerprint may be formed using two or more genes and measuring the amount of their transcripts in a cancer sample wherein the measured, transcription pattern reflect the type or stage of cancer.

### BRIEF DESCRIPTION OF THE FIGURES

FIGS. 1A-1I show a real-time quantitative RT-PCR quantitation of gene expression in mouse TRAMP tumors. Total RNA and cDNA was prepared from two normal prostate (Normal 1 and Normal 2) and two TRAMP tumors (TRAMP 1 and TRAMP 2). The level of gene expression in the cDNA was measured in the presence of its respective primer and SYBR Green I Dye in real-time quantitative PCR. PCR reactions for each sample were performed in duplicate for both target gene and cyclophilin normalizer. The level of gene expression was calculated after normalizing against the cyclophilin level in each sample and presented as relative units in the graph.

FIGS. 2A-2C show induction of neovascularization in nude mouse skin by SK-MEL-2 human melanoma VEGF-transfectants. Human SK-MEL-2 melanoma cells were transfected with human VEGF₁₆₅ under the direction of a CMV promoter and clones (hVEGF-Low and hVEGF-High) were isolated. A. Northern blot detection of VEGF expression in hVEGF-Low B and hVEGF-High clones. The fold increase of hVEGF-High over hVEGF-Low was calculated after normalization to the β-actin. B. Real-time quantitative RT-PCR detection of absolute VEGF copy numbers in VEGF-Low and VEGF-High clones. C. Angiogenesis in nude mouse skin was induced by subdermal injection of 0.25 cc Matrigel containing 1.5×10⁶ transfected cells as indicated or no cells (control). Tissue was harvested and photographed at day 6.

FIG. 3 shows a real-time quantitative RT-PCR quantitation of an absolute mRNA copy number in skin specimens from nude mice injected with Matrigel±hVEGF-transfectants. Upon dissection, Matrigel was removed and total RNA and cDNA was prepared from the overlying skin associated with control Matrigel, Matrigel+hVEGF-Low transfectants, and Matrigel+hVEGF-High transfectants. The precisely measured and 10-fold serially diluted cDNAs templates (Ang-1, Ang-2, Flt-1, KDR, PECAM, VE-Cadherin, Tie-1, and Tie-2) were used as external standard curve. The copy numbers of each mRNA was calculated after normalizing to million copies of cyclophilin in each sample and data are presented as copy number/million cyclophilin mRNA molecules.

### DETAILED DESCRIPTION OF THE INVENTION

The present invention provides a method for determining an absolute copy number of a transcript or a plurality of transcripts of interest in a biological specimen.

The method of the present invention is useful as a screening, diagnostic and prognostic tool in diseases wherein gene transcription patterns are known to change or fluctuate. The method is also useful for pharmacogenomic applications to, for example determine customized medication, for pre-clinical and clinical trials which use pharmaceuticals that are targeted to affect gene expression as an efficacy measurement tool and wherein measurement of transcript levels is an important indicator of effectiveness of the pharmaceuticals. The method can further be used to monitor the efficacy of a treatment in a patient.

The “first standard” as used herein refers to a control vector, which is used to normalize the sample size or the amount of cells used in the quantification reaction. To prepare such standard, a coding region of a nucleic acid, or cDNA, encoding a housekeeping gene, or a part of the coding region of the housekeeping gene is first cloned into a vector backbone. Such “housekeeping vector” is then serially diluted into at least two, preferably three, four or five, alternatively up to 10-20 serial dilutions using a buffer that is suitable for PCR. Such buffers are well known to one skilled in the art.

In one preferred embodiment, the first and second standards are comprised within one vector, referred herein as a “dual”, “combination” or “double” standard vector. Such combination standard vector comprises an amplifiable nucleic acid fragment of at least one housekeeping gene and an amplifiable nucleic acid fragment of at least one gene of interest cloned into the same vector backbone. Consequently, only one standard dilution set as described above, referred herein as a “dual standard” is needed for the quantification reactions.

A housekeeping gene is generally defined as a gene that is expressed in all cells because it provides a basic function needed in all cell types. The term “housekeeping gene” referred to herein includes also any transcript the amount of which does not change or fluctuate during a pathological condition of interest. Typical examples of such transcripts include, but are not limited to constitutively expressed, or housekeeping gene transcripts such as cyclophilin, 18S ribosomal RNA, beta-actin, glyseraldehyde 3-phosphate dehydrogenase (GADPH), gelsolin, porphobilinogen deaminase, or beta-2 microglobulin. A housekeeping gene is selected based upon knowledge of the pathological condition of interest. For example, in case of diagnosing cancer, such as prostate adenocarcinoma or colon cancer, or other pathological condition related to abnormal angiogenesis, cyclophilin can be used as a housekeeping gene. Sequences of different housekeeping genes are readily available in the existing databases, such as GenBank.

The terms “sufficient portion” or “amplifiable fragment” as used herein refer to the size of the housekeeping gene or gene of interest, or a fragment thereof and may vary from at least about 50 base pairs (bp) to about 5,000-10,000 base pairs (bps). Preferably, the housekeeping gene or the gene of interest or fragment thereof that is cloned into a vector backbone is about 70-140 bp, most preferably the fragment is about 120 bp. The fragments may be cloned to the vector using traditional cloning methods, PCR or preparing the fragments using a nucleic acid synthesizer and then cloning them into a vector.

The term “vector” or “vector backbone” according to the present invention includes any plasmid, cosmid, artificial chromosome, viral vector and the like, which can be used to clone a nucleic acid into. Such vectors are known to one skilled in the art In one preferred embodiment, the vector backbone is a plasmid, for example, PCRScript vector (Stratagene, La Jolla, Calif.).

The vector backbone comprising the housekeeping gene coding region or a fragment thereof is used to transform a host cell. The host cell can be a procaryotic or a eukaryotic cell including, but not limited to bacteria and yeast and mammalian cells. Preferably the host cell is a bacterium, most preferably a suitable E. coli strain such as XL1-Blue E. coli. The host cell is transformed with the vector according to protocols known to one skilled in the art (see e.g. the procedures disclosed in Molecular Cloning: A Laboratory Manual 3rd Ed., Sambrook and Russel, Cold Spring Harbor Laboratory Press, 2001). The plasmid DNA from the host cells is isolated and the optic density (OD) measured to determine the copy number of the vector the molecular weight of which is known.

The recombinant vector with amplifiable fragment of at least one housekeeping gene or fragment thereof is thereafter serially diluted in a buffer to about 3-20 different serial dilutions, preferably, four to five dilutions. Buffer can be any PCR suitable buffer, which are known to one skilled in the art. For example, samples with 1, 10, 100, 10³, 10⁴, 10⁵, 10⁶, 10⁷ copies of the vector per micro liter of a buffer in the dilution series can be used. This dilution series, serves as a standard referred to herein as the “first standard”, which is used to determine the “size” of the tissue sample of interest The term “size” herein means the relative amount of the staring material which could be otherwise determined, for example, by weight of the sample or the number of the cells in the sample. In the present invention, the sample size is determined by using the copy number of a housekeeping gene transcript as a measuring point to which the copy number of the gene of interest transcript can then be compared to.

The gene of interest is any gene or set of genes the expression of which changes during development, or before, during or after a pathological condition such as a benign or malignant tumor, a neurological disorder, heart disease, renal disease, or an autoimmune disorder, or genes whose expression changes as a response to organic or inorganic molecules administered into a subject cells, tissues, or animals, including humans, as whole. Genes of interest include, for example, genes regulating angiogenesis, such as angiopoietin-1 and -2, endostatin, angiostatin, receptor tyrosine kinases Tie-1 and -2, the family of vascular endothelial growth factors (VEGFs) and their receptors (VEGFRs); genes regulating apoptosis; genes regulating cytokine expression such as interleukin, interferons, and their receptors and downstream signaling molecules. Numerous genes that can be used to diagnose a disorder or progression of a disease are known to one skilled in the art, and the realm of genes associated with diseases, disorders, and development is constantly increasing.

After selecting the gene(s) of interest a nucleic acids encoding such gene(s) or any part of the coding region of the gene or cDNA is cloned into a vector to create a “second standard.” The size of the gene of interest cDNA is preferably between about 100 bp up to about 5-10 kbp. This vector is processed the same way as the “first standard” described above to create a serial dilution of the vector. This dilution series serves as a standard, which is used to determine the efficacy of the PCR reaction. The efficacy of the PCR reaction is known to be dependent upon the size and sequence of the amplicon and the size and sequence of the primers. Therefore, to accurately determine the copy number of any transcript of interest, a normalizing standard with the gene of interest sequence must be used.

In general, many factors influence the amplification success of a pair of primers. Some of the properties of primers which can affect the outcome of PCR include: the GC/AT ratio, length, melting temperature, and the extent of annealing between primers. The location of a primer also heavily influences its usefulness. The primers for amplifying the first and second normalizing standard as well as the transcript of interest in a biological sample are determined using, for example, a computer program designed for primer design, such as Primer Express (Applied Biosystem, Foster City, Calif.), Primer Premier 5 (Biosoft International, Palo Alto Calif.), Primer3 (Steve Rozen, Helen J. Skaletsky (1998) Primer3. Code available at http://www-genome.wi.mit.edu/genome_software/other/primer3.html) and GeneFisher (“GeneFisher—Software Support for the Detection of Postulated Genes”, R. Giegerich, F. Meyer, C. Schleiermacher, ISMB-96, Proceedings of the Fourth International Conference on Intelligent Systems for Molecular Biology, AAAI Press (ISSN 57735-002-2)).

In general, the size of the amplicon may be from about 50 bp to about 5 kbp, preferably the amplicon is about 50-600 bp, most preferably about 80-120 bp. The phrase “size of the amplicon” refers to the size of the nucleic acids the PCR primers are designed to amplify.

Using the first and second standards or the dual standard, and primers designed to amplify the standards, the method can be applied to determine an absolute copy number of any transcript or transcript combination in a biological specimen as described.

The “sample cells”, “sample tissue” or “biological specimen” as used in the specification refer to any tissue sample which contains cells with DNA including, for example, cell culture sample, a blood sample, a tissue biopsy sample, a buccal sample, and a feces sample.

Total or mRNA can be isolated from the sample by using any conventional RNA isolation method known to one skilled in the art (see e.g. the procedures disclosed in Molecular Cloning: A Laboratory Manual 3rd Ed., Sambrook and Russel, Cold Spring Harbor Laboratory Press, 2001). Preferably, RNA is isolated using a cell lysis buffer that is compatible with an RT-PCR buffer, and that combines both RNase inactivating chemistry and DNaseI treatment (see, e.g. Cells-to-DNA system by Ambion, Inc. Austin, Tex.). A random RT-PCR using random primers is preformed using the isolated total RNA or mRNA to prepare cDNA sample comprising cDNA from all the transcripts present in the RNA sample. To allow accurate detection of transcript levels, it is important to destroy genomic DNA from the biological specimen before PCR amplification. Alternatively, the primers may be designed to amplify target genes so that the primer sequence covers sequences in two exons separated by intronic sequence in the genomic DNA,

For example, real-time PCR may be used. In September 1993 Higuchi et al. published a report in the journal Biotechnology (NY). Titled “Kinetic PCR analysis: real-time monitoring of DNA amplification reactions,” the paper described the technique that would come to be known as Real-Time PCR. In the original report, the authors used the DNA binding properties of the ethidium bromide (EtBr) to monitor the accumulation of PCR product in a reaction tube. Currently there are a number of different ways to detect PCR product in Real-Time PCR analysis. Primers that have been labeled with fluorogenic tags, as well as new, more sensitive DNA dyes, are now common (for review, see e.g. http://www.biocompare.com/spotlight.asp?id=133).

Several systems are currently available for performing real-time PCR and include, but are not limited to the ABI PRISM® 7000 and The ABI PRISM® 7900HT Sequence Detection System (Applied BioSystems), iCycler iQ Real-Time PCR Detection System (Bio-Rad), Smart Cycler® System and Smart Cycler® TD System (Cepheid), R.A.P.I.D.™ System (Idaho Technology Inc.), Rotor-Gene 3000 Real-Time DNA Amplification System (Phenix Research Products), LightCycler® Instrument (Roche Applied Science), and Mx3000P™ Real-Time PCR System and Mx4000™ Multiplex Quantitative PCR System (Stratagene).

An example of a real-time PCR method useful according to the present invention is TAQMAN® PCR. TAQMAN® PCR is performed from the sample cDNA using primer sets designed for the first standard, amplifying the housekeeping gene and the second standard, by amplifying the gene of interest (ABI 7700 (TAQMAN®), Applied BioSystems, Foster City, Calif.). A PCR, for example, TAQMAN® PCR is performed also from the first and second normalizing standards, or the dual standard in case both the housekeeping gene(s) and gene(s) of interest are cloned into the same vector backbone.

The basis for PCR quantitation using the ABI 7700 instrument is to continuously measure PCR product accumulation using a dual-labeled flourogenic oligonucleotide probe called a TaqMan® probe. This probe is composed of a short (ca. 20-25 bases) oligodeoxynucleotide that is labeled with two different fluorescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two flourophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity. The ABI Prism 7700 uses fiber optic systems which connect to each well in a 96-well PCR tray format . The laser light source excites each well and a CCD camera measures the fluorescence spectrum and intensity from each well to generate real-time data during PCR amplification. The ABI 7700 Prism software examines the fluorescence intensity of reporter and quencher dyes and calculates the increase in normalized reporter emission intensity over the course of the amplification. The results are then plotted versus time, represented by cycle number, to produce a continuous measure of PCR amplification. To provide precise quantification of initial target in each PCR reaction, the amplification plot is examined at a point during the early log phase of product accumulation. This is accomplished by assigning a fluorescence threshold above background and determining the time point at which each sample's amplification plot reaches the threshold (defined as the threshold cycle number or CT). Differences in threshold cycle number are used to quantify the relative amount of PCR target contained within each tube as described previously.

The PCR reactions using the different primers may be performed either in the same reaction mixture or in parallel reaction mixtures. It is preferred that the standard PCRs and the PCR on the RNA of the biological specimen are performed in separate reactions.

When the TAQMAN® PCR is performed, the first standard curve is prepared from the first standard using cyclo threshold (CT) from dilution samples with different copy numbers of the first vector (copy number on a y-axis and CT on an x-axis). The copy number of the housekeeping gene in the tissue sample is extrapolated from the plot using the CT obtained from the tissue sample PCR. In general, the CT reflects the amount of the PCR amplification product and can be obtained using any method known to one skilled in the art for quantitating PCR products. Labels, such as radioactivity and dyes, for example, fluorescent dyes can be used to label the primers. Likewise, a second standard curve is prepared from the second standard using CT from dilution samples with different copy numbers of the second vector (copy number on a y-axis and CT on an x-axis). The copy number of the gene of interest in the tissue sample is extrapolated from the plot using the CT obtained from the tissue sample PCR for both the housekeeping gene and the gene of interest.

The absolute copy number of the gene of interest transcripts in the biological specimen of interest can then be determined by taking the copy number of the gene of interest extrapolated from the second standard curve and dividing it by the copy number of the housekeeping gene extrapolated from the first standard curve. This number reflects the actual, absolute amount of the gene of interest in the tissue sample.

Preparation of a Screening and Diagnostic Fingerprint Standard

A pathological sample collection is prepared using tissue samples from several pathological samples in different pathological stages and obtaining a gene expression pattern, or a “fingerprint”, of the samples using the method described above. Analysis of several genes of interest will result in a panel for informative markers specific for a disease condition or a disease stage and allow diagnosis of a sample from a subject of interest.

Similar approach can be used to prepare a fingerprint for screening large populations for susceptibility to certain diseases such as diabetes, heat diseases, a variety of cancers and the like.

A tissue sample from a subject of interest can then be prepared and the gene expression pattern of the sample can be compared to the gene expression pattern of the pathological sample collection.

Database of Screening/Diagnostic Fingerprints

A disease or disease stage specific “fingerprint” comprising the absolute copy number of a set of genes in a specific pathological condition can be used to create a computer database comprising the fingerprint pattern and corresponding diagnosis, prognosis, and treatment information. When a fingerprint of a biological sample is created it will be compared to the database of known fingerprints to assist in making a diagnostic, prognostic determination or treatment suggestion.

It is to be understood that while the invention has been described in conjunction with the preferred specific embodiments thereof that the foregoing description as well as the examples that follow are intended to illustrate and not limit the scope of the invention. Other aspects, advantages and modification within the scope of the invention will be apparent to those skilled in the art to which the invention pertains.

### EXAMPLE 1

Plasmid Construction for Normalizing Standards (First and Second Standard Vector)

A PCR primer covering an entire region of real-time PCR amplicon of the housekeeping transcript or the gene of interest transcript coding region will be designed. A real-time PCR amplicon is typically short, and usually it is around 80-120 bp long. PCR amplicon will normally be larger, usually about 400-600 bp.

PCR is performed using a cDNA library and the PCR product will be inserted into a vector, such as PCRScript from (Stratagene, Palo Alto, Calif.).

The XL1-Blue E. coli bacterial strain will be transformed with the ligated plasmid and one colony which contains the plasmid with the cDNA insert will be picked. The colony with the plasmid will be amplified and the plasmid will be isolated and the optic density (OD₂₆₀) of the plasmid DNA will be measured, if necessary, the plasmid will be diluted in dH₂O to measure the OD₂₆₀. The OD₂₆₀ value is used to calculate the plasmid concentration (μg/ml). Because the number of bases in the plasmid is known, the molecular weight of the plasmid can be counted (molecular weight (mw) of one base pair about 638 daltons, therefore, plasmid mw equals to the number of bases times 638; mw of each plasmid equals to the plasmid mw/6×10²³). The plasmid concentration (μg/ml) will then be converted into plasmid density (number of plasmids/mi).

Measurement of the House Keeping Gene Copy Numbers in Samples of Interest

Total RNA from cells or tissues will be isolated and reverse transcribe it into cDNA.

The real-time PCR reagent (real-time PCR primers for the house keeping gene, Taq polymerase and dNTP) will be mixed with the 3 μl precisely quantified and 10-fold serially diluted plasmid which contains the house keeping gene cDNA or the 3 μl cDNA prepared from samples of interest.

30 μl of each sample mixture will be added into a 96-well plate. The 10-fold serially diluted plasmid samples will be added in the first row of the 96-well plate (4 serially diluted plasmid in triplicates) and cDNA samples in the other wells (triplicates).

The plasmid copy numbers are input when a plate is set up in real-time PCR machine. The machine will then automatically plot a standard curve in the end of the cycle (Cycle threshold (CT) in x-axis and Copy number in y-axis). All the sample cDNAs will have a CT value after each real-time PCR analysis and the machine will automatically plot the CTb number (x-value) with the standard curve and provide copy numbers of housekeeping gene.

At least two, preferably three to five, 10-fold serial dilutions of the plasmid preparation will be prepared and a sample of 3 μl of them is used in a real-time PCR. Also, 3 μl of the cDNA samples will be used in the real-time PCR. In addition to the sample cDNAs or plasmids, the PCR reaction mixture comprises Taq polymerase, dNTP mix, and the appropriately labeled real-time PCR primers. The PCR reaction is performed on a 96-well plate with about 30 μl reaction mix. The plasmid samples are added in the first row of the plate.

Measurement of the Gene of Interest Copy Numbers in Samples of Interest

Real-time PCR reagent (real-time PCR primers for the gene of interest, Taq polymerase and dNTP) will be mixed with the 3 μl of precisely quantified and 10-fold serially diluted plasmid which comprises the gene of interest cDNA or the 3 ul cDNA prepared from samples of interest.

30 ul of each sample mixture will be added into a 96-well plate. The 10-fold serially diluted plasmid samples will be added into wells in the first row of the 96-well plate (4 serially diluted plasmid in triplicate) and the cDNA samples for the biological sample will be added into the other wells (triplicates).

The plasmid copy numbers are input when set up a plate in real-time PCR machine, so the machine will automatically plot a standard curve when finishes running (Cycle threshold (CT) in x-axis and Copy number in y-axis). All the sample cDNAs will have a CT value after each real-time PCR analysis and machine will automatically plot the CT number (x value) with the standard curve and provide a copy numbers of gene of interest.

Calculate Copy Number of Gene of Interest in Every Million Copies of House Keeping Gene

Each sample of interest, we used same volume (3 ul) to measure house keeping gene or gene of interest. Therefore, the number of copies of gene of interest present can be calculated if there are a million copies of house keeping gene.

When copy numbers of a panel of genes from a number of samples is measured, a single and simple bar graph (gene of interest/10⁶ house keeping gene on a y-axis and different gene names on an x-axis) will be drawn. This graph or “fingerprint pattern” will be the source of information for a disease progression or an experimental treatment status.

### EXAMPLE 2

We have developed a sensitive, simple and widely applicable assay to 1) measure copy numbers of specific mRNAs using real-time quantitative RT-PCR, and 2) identify a profile of gene expression closely associated with angiogenesis. We measured a panel of nine potential angiogenesis markers from a mouse transgenic model of prostate adenocarcinoma I ) and a mouse skin model of vascular endothelial growth factor (VEGF) driven angiogenesis. In both models, expression of VEGF correlated with expression of mRNAs encoding other angiogenic cytokines (angiopoietin-1 and angiopoietin-2), endothelial cell receptor tyrosine kinases (Flt-1, KDR, Tie1), and endothelial cell adhesion molecules (VE-Cadherin, PECAM-1). Relative to control, in dermis highly stimulated by VEGF, the Ang-2 mRNA transcript numbers increased 35-fold, PECAM-1 and VE-cadherin increased 10-fold, Tie-1 increased 8-fold, KDR and Flt-1 each increased 4-fold, and Ang-1 increased 2-fold. All transcript numbers were correspondingly reduced in skin with less VEGF expression, indicating a relationship of each of these seven markers with VEGF. Thus, this study identifies a highly efficient method for precise quantification of a panel of seven specific mRNAs that correlate with VEGF expression and VEGF-induced neovascularization, and it provides evidence that real-time quantitative RT-PCR offers a highly sensitive strategy for monitoring angiogenesis.

Angiogenesis, the growth of new blood vessels, is widely regarded as an attractive target for controlling cancer and other important pathologies including retinopathy. Angiogenesis occurs early in tumor progression.^{1,2} Moreover, angiogenesis correlates not only with the onset of tumor development but also with growth and invasion of established tumors.^{1,3} Consequently, tools for quantifying angiogenesis are valuable for molecular profiling of tumors, monitoring the status of tumor progression and estimating the malignant potential of established tumors. In addition, molecular quantification of angiogenesis would be useful for the study of a variety of experimental models of pathologic angiogenesis including retinopathy.

The most widely used technique to assess angiogenesis in clinical settings relies on immunohistochemical staining of blood vessels in fixed biopsies of specimens. Even with the use of sophisticated computerized imaging systems, this technique is labor-intensive and cannot be used as a quick and quantitative screening procedure. Individual vascular markers have been studied by quantitative and semi-quantitative techniques usually in a single tissue, but there has been little work on a comprehensive assay suitable for a varying degrees of angiogenesis. Thus, there is a need to develop a sensitive molecular assay, that allows molecular profiling of new vessel growth and that can be used to monitor angiogenesis and the efficacy of chemopreventive and other drug-based therapies towards suppressing angiogenesis.

Real-time quantitative (kinetic) PCR technology offers high sensitivity and high throughput capacity.⁴⁻⁶ This technique uses SYBR Green I dye to detect and verify product by melting curve analysis, and is applicable for validation of samples containing cDNA fragments prepared with gene-specific primers.⁴⁻⁶ The PCR amplification and detection in real-time quantitative RT-PCR occur simultaneously, thereby avoiding the need for post-PCR analysis and minimizing the risk of carryover contamination. Furthermore, this technique is highly reproducible over a wide dynamic range, and permits simultaneous quantitative analysis of a large number of samples with varying input concentrations allowing for statistically significant data. Utilizing real-time quantitative PCR technology and precisely quantified external cDNA templates as standards, the TAQMAN® system (Applied Biosystems) is capable of detecting copy numbers of nucleic acid targets with sensitivity as low as 10 copies with optimal detection from 100-500 copies. Recent studies have applied the technique to measure copy numbers of target genes relative to cell number⁷ or total RNA.⁸ However, a method for precise standardization is required to accurately and reproducibly measure mRNA copy number from different sources.

Therefore, in this study we employed real-time quantitative RT-PCR in combination with specific cDNA standards to determine absolute mRNA copy numbers. Since it is likely that no single mRNA marker is predictive of the degree of angiogenesis in all tumor types and all tissues, we evaluated a panel of potential markers. Such markers include those directly correlated with endothelial cell number and/or mRNAs encoding proteins directly associated with the process of neovascularization. VE-cadherin, an adhesion receptor specific to endothelium, is one candidate for which the expression level would be expected to correlate with endothelial cell number.^{9,10} Other candidates, expressed in relative abundance by endothelial cells in comparison with other cell types, include those encoding the adhesive protein PECAM-1 ^{11,12} and the receptor tyrosine kinases Flt-1, KDR, Tie-1 and Tie-2.^{13,18} Finally, VEGF^{14,19,20} and two other cytokines associated with angiogenesis, angiopoietin-1 (Ang-1) and angiopoietin-2 (Ang-2),²¹⁻²⁴ were included in our analyses.

Two independent animal models served as sources of tissue for testing the correlation of the marker panel with angiogenesis. One of them is a mouse transgenic model of prostate adenocarcinoma (TRAMP), in which spontaneous prostate intraepithelial neoplasia (PIN) develop at about 6-12 weeks of age and progress to invasive carcinoma and neoplasia between 18 and 24 weeks of age. Tumor progression and histological features of carcinomas in this animal model highly resemble human prostate carcinoma In the TRAMP model, the probasin promoter drives the expression of SV40 Tag in the prostate causing a functional inactivation of P53 and Rb tumor suppressor genes.²⁵ Tumors are highly vascularized²⁶ and metastatic²⁷ and therefore, TRAMP mice constitute an excellent model to study angiogenic factors. The other is a mouse Matrigel model of skin angiogenesis in which neovascularization is provoked by subcutaneous implantation of Matrigel containing human VEGF-transfected human melanoma cells. Real time RT-PCR data from both models identified the same panel of mRNAs which corresponded to both VEGF expression and VEGF-induced neovascularization. Overall, this study indicates that real-time RT-PCR offers a sensitive new approach for molecular profiling of a panel of mRNA markers that is closely associated with tumor neovascularization.

Skin Angiogenesis Model

Athymic NCr nude mice (7-8 week old, females) were injected subdermally midway on the right and left back sides with 0.25 ml Matrigel (BD Biosciences) at a final concentration of 10 mg/ml protein, together with 1.5×10⁶ SK-MEL-2 cells tnansfected with pcDNA3.1 (Invitrogen) containing a human VEGF₁₆₅ insert under the direction of the CMV promoter. To provoke different intensities of neovascularization, two clonal transfectants expressing different levels of VEGF were isolated and characterized (see Results). Soon after injection, the Matrigel implant solidified, and persisted without apparent deterioration throughout the 6-day assay interval. After six days, the animals were euthanized, and specimens harvested by dissection.

TRAMP mice. The C57BL/6 male TRAMP mice were obtained from Jackson Laboratories and were bred and tested positive by slot blot. Animals were observed weekly for tumor appearance and prostate tumors were removed at age of 8-9 months. Normal prostates were obtained from C57BL/6 non-transgenic male mice at the same age. Part of each tumor was frozen in liquid nitrogen before RNA extraction and the rest was immersed in a 4% paraformaldehyde solution for histological analysis. Fixed samples were dehydrated through ethanol and xylene, embedded in paraffin, sectioned (4 μm in thickness) and stained with hematoxylin and eosin.

RNA Isolation and cDNA Preparation. Total RNA was extracted by using RNeasy RNA extraction kit (Qiagen, Chatsworth, Calif.). Briefly, tissues were lysed in guanadinium isothiocyanate buffer, and RNA was purified following manufacturer's instruction. The purified RNA was suspended in DEPC treated H₂O. To generate cDNA, 1 ug total RNA was treated with DNase I (Ambion, Austin, Tex.) to remove any contaminating genomic DNA. The DNase-treated RNA (100 ng) was then converted into cDNA by using murine leukemia virus reverse transcriptase (Gibco BRL Life Technologies, Bethesda, Md.). All cDNA samples were aliquoted and stored at −80° C.

Northern Analyses. Northern blotting was performed as described previously.²⁸ 10 ug of total RNA was used from each sample and hybridization was carried out overnight at 65° C. with α—³²P-dCTP-labeled human VEGF165 (823 bp fragment encompassing the coding region and 330 bp of 3′UTR) and control β-actin cDNA. Probes were prepared by the random-primed synthesis method using the Multiprime Kit (Amersham Corp., Arlington Heights, Ill.). Blots were washed at high stringency (1% SDS and 1×SSC at 55° C.) and exposed to Kodak MR film.

TAQMAN® Quantitative Real Time RT-PCR

Real-time quantitative RT-PCR primers targeting murine VEGF, Flt-1, KDR, Tie-1, Tie-2, Ang-1, Ang-2, PECAM, VE-Cadherin, and cyclophilin were designed by using Primer Express software (Applied BioSystems, Foster City, Calif.) and sequences are listed in Table 1 (5′ to 3′). Specificity of each primer to the sequence of choice was checked by NCBI Blast module and was synthesized by Genemed Synthesis (South San Francisco, Calif.). To assure the specificity of each primer set, amplicons generated from PCR reactions were analyzed for specific melting point temperatures by using the first derivative primer melting curve software supplied by Applied BioSystems. The SYBR Green I assay and the ABI Prism 7700 Sequence Detection System (Applied Biosystems) were used for detecting real-time quantitative PCR products from 0.25-2.5 ng reverse transcribed cDNA samples and performed as described previously.²⁹ SYBR Green I dye intercalation into the minor groove of double-stranded DNA reaches an emission maximum at 530 nm. PCR reactions for each sample were done in duplicate for both target gene and cyclophilin control. The level of target gene expression was calculated following normalization of the cyclophilin level in each sample and presented as relative units.

TABLE 1
TAQMAN ® Primer Sequences

| | |
|---|---|
| Gene | Forward [SEQ ID Nos: 1-10] |

| | |
|---|---|
| Ang-1 | 329-CATTCTTCGCTGCCATTCTG |
| | [SEQ ID NO: 1] |
| Ang-2 | 1478-TTAGCACAAAGGATTCGGACAAT |
| | [SEQ ID NO: 2] |
| Cyclophilin | 5-CAGACGCCACTGTCGCTTT |
| | [SEQ ID NO: 3] |
| Flt-1 | 2421-GAGGAGGATGAGGGTGTCTATAGGT |
| | [SEQ ID NO: 4] |
| KDR | 4570-GCCCTGCTGTGGTCTCACTAC |
| | [SEQ ID NO: 5] |
| PECAM | 1652-GAGCCCAATCACGTTTCAGTTT |
| | [SEQ ID NO: 6] |
| Tie-1 | 742-CAAGGTCACACACACGGTGAA |
| | [SEQ ID NO: 7] |
| Tie-2 | 1804-ATGTGGAAGTCGAGAGGCGAT |
| | [SEQ ID NO: 8] |
| VE- | 1853-TCCTCTGCATCCTCACTATCACA |
| Cadherin | [SEQ ID NO: 9] |
| VEGF | 805-GGAGATCCTTCGAGGAGCACTT |
| | [SEQ ID NO: 10] |

| | |
|---|---|
| Gene | Reverse [SEQ ID Nos: 11-20] |

| | |
|---|---|
| Ang-1 | 431-GCACATTGCCCATGTTGAATC |
| | [SEQ ID NO: 11] |
| Ang-2 | 1598-TTTTGTGGGTAGTACTGTCCATTCA |
| | [SEQ ID NO: 12] |
| Cyclophilin | 137-TGTCTTTGGAACTTTGTCTGAA |
| | [SEQ ID NO: 13] |
| Flt-1 | 2536-GTGATCAGCTCCAGGTTTGACTT |
| | [SEQ ID NO: 14] |
| KDR | 4632-CAAAGCATTGCCCATTCGAT |
| | [SEQ ID NO: 15] |
| PECAM | 1769-TCCTTCCTGCTTCTTGCTAGCT |
| | [SEQ ID NO: 16] |
| Tie-1 | 863-GCCAGTCTAGGGTATTGAAGTAGGA |
| | [SEQ ID NO: 17] |
| Tie-2 | 2081-CGAATAGCCATCCACTATTGTCC |
| | [SEQ ID NO: 18] |
| VE- | 1974-GTAAGTGACCAACTGCTCGTGAAT |
| Cadherin | [SEQ ID NO: 19] |
| VEGF | 933-GGCGATTTAGCAGGAGATATAAGAA |
| | [SEQ ID NO: 20] |

The numbers flanking the sequences in the Table 1 refer to the position in the coding sequences of the named genes as shown in the sequences in the GenBank database.

cDNA template construction for absolute quantitation of specific mRNAs. For each of nine genes studied, we cloned cDNA that covers the region of real-time quantitative RT-PCR primers and inserted the cDNA into PCRScript vector (Strategene, La Jolla, Calif.). To use as an external standard for real-time quantitative PCR detection, each cDNA construct was purified, precisely quantified and 10-fold serially diluted to 10 copies per microliter. SYBR Green I provided reproducible detection of the cDNA template that ranged from less than 10 copies per reaction to more than 10⁷ copies per reaction. In each real-time quantitative PCR assay, a 10-fold serially diluted cDNA template series was added to measure standard curve for copy number. Each sample was analyzed in triplicate and copy numbers determined from each corresponding standard curve. Each gene was then normalized to 10⁶ copies of cyclophilin control and data presented as copy numbers/10⁶ cyclophilin copies.

Results

In order to measure quantitatively the level of gene expression in cDNA samples, specific TAQMAN® PCR primers were designed for each potential angiogenesis marker listed in Table 1. The specificity of each primer set was validated by analyzing the melting point temperature of each amplicon in the ABI Prism 7700 Sequence Detection System. Cyclophilin, a protein known as a primary cytosolic receptor for cyclosporine A, is abundant (0.05% to 0.4% of total protein) in the cytosol and is ubiquitous in cells and tissues of eukaryotic organisms.³⁰ The expression level of cyclophilin was constant in both TRAMP and skin samples in comparison with 18S rRNA (data not shown). Since the level of 18S rRNA transcript is almost 21¹² to 2³⁰ times higher than most mRNAs in real-time quantitative real-time quantitative RT-PCR detection, it is difficult to use 18S rRNA as a housekeeping gene and accurately calculate the target gene expression. Therefore, cyclophilin mRNA was used as the internal control gene in our real-time quantitative RT-PCR studies.

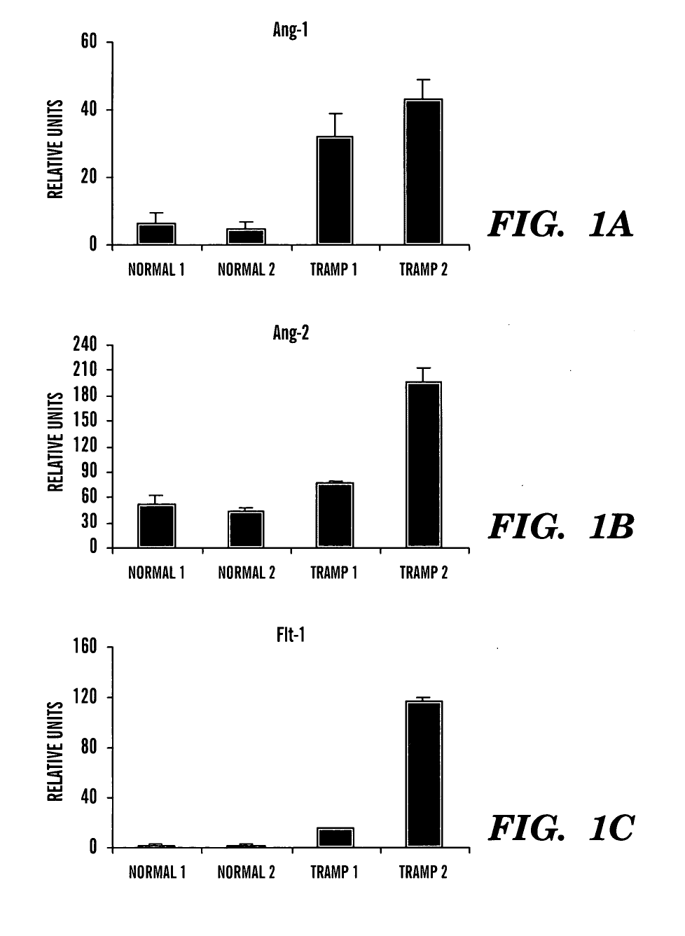

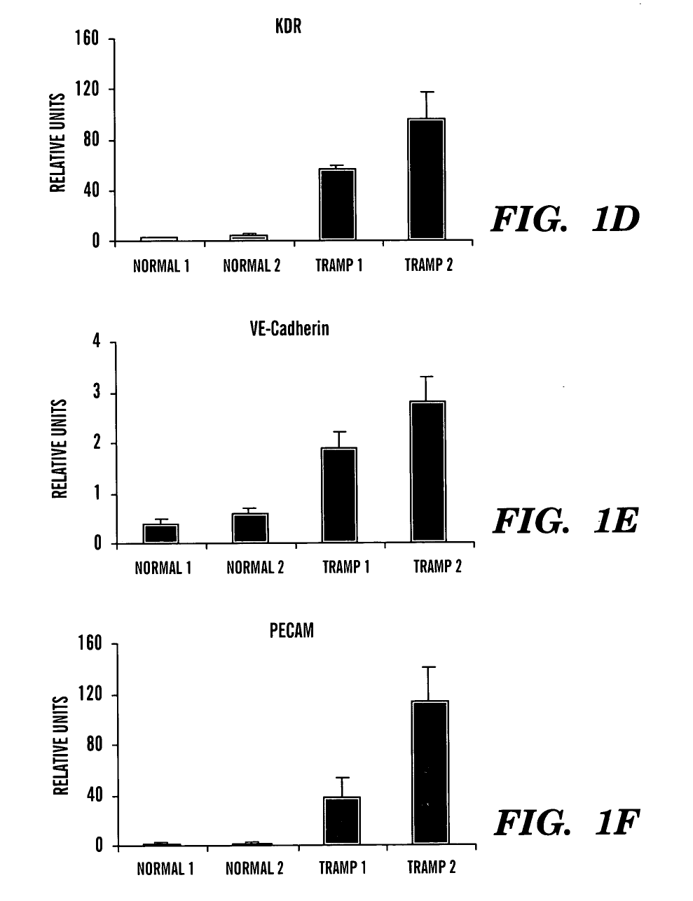

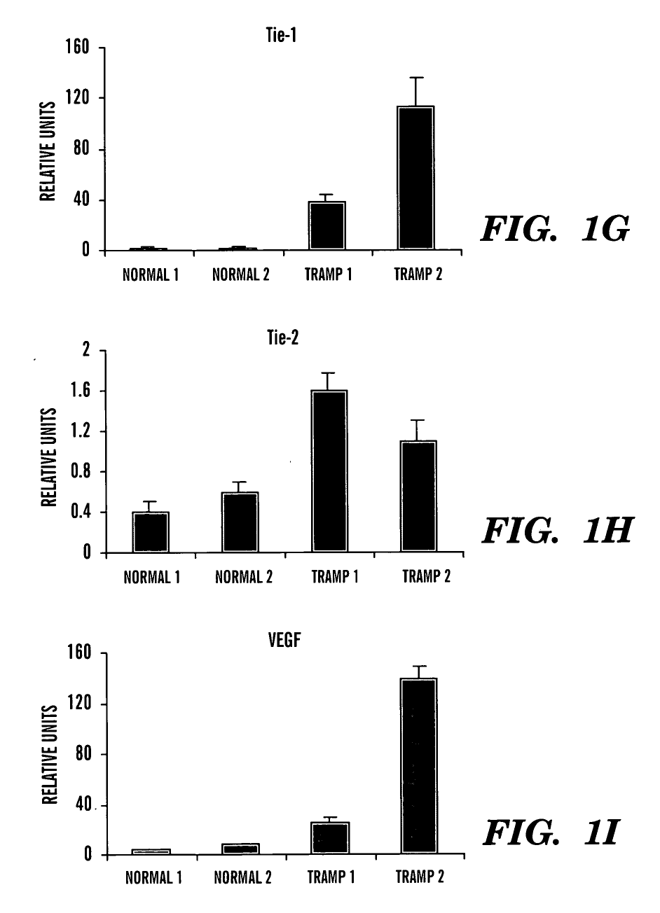

First, to identify potential tumor angiogenesis markers, total RNA was isolated from two normal and two TRAMP C57 mice prostates and was reverse transcribed into cDNA. Tumor TRAMP1 was isolated at 8 months of age and was 1 cm³ in size, whereas tumor TRAMP2 was isolated at 9 months of age and was nearly double the size of TRAMP1. The histological analysis for both tumors revealed the presence of advanced prostate adenocarcinomas with the characteristics described previously. Neoplastic cells were relatively small and showed hyperchromatic nuclei and small amount of cytoplasm. Both tumors also showed high vascularization and areas of necrosis. Relative to normal prostate, both TRAMP1 and TRAMP2 tumors expressed higher levels of all eight angiogenesis associated mRNAs listed in Table 1 (FIG. 1). Interestingly, the TRAMP2 tumor, whose VEGF expression was 3.7-fold higher than TRAMP1 tumor, also expressed higher levels of Ang-1, Ang-2, Flt-1, KDR, PECAM, Tie-1 and VE-Cadherin mRNAs. Tie-2 expression did not follow the same trend, although Tie-2 expression was higher in both TRAMP tumors as compared with normal prostate controls.

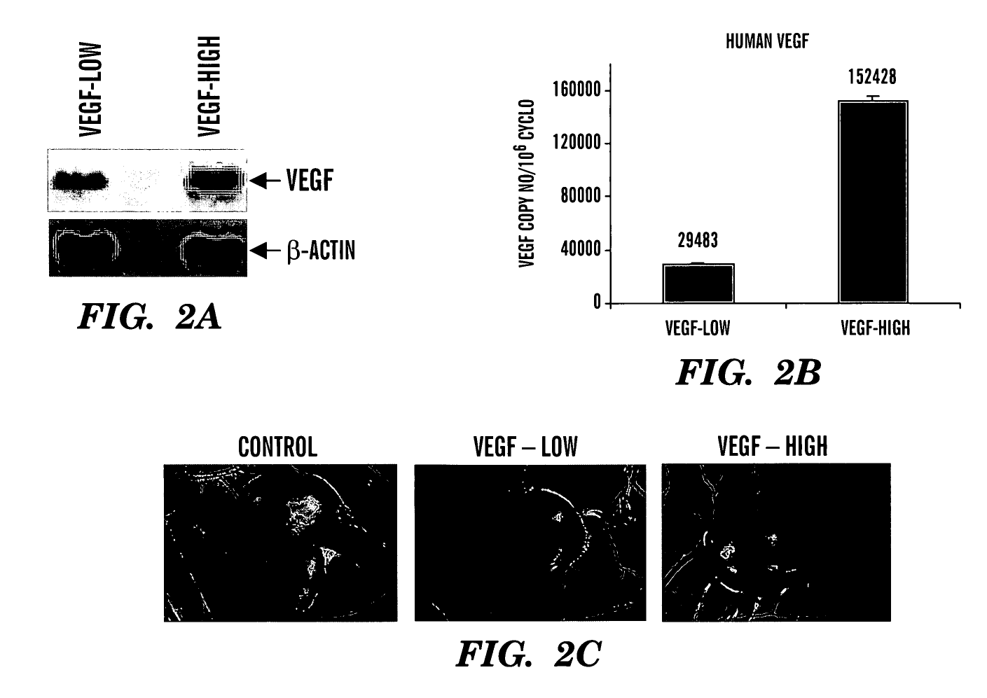

In order to precisely measure the expression of the candidate marker mRNAs identified above in relation to VEGF expression, we transfected human SK-MEL-2 melanoma cells with a human VEGF₁₆₅ cDNA construct and isolated two clones expressing different levels of human VEGF mRNA (FIG. 2). To determine absolute mRNA numbers, we cloned and isolated individual cDNA templates that cover the sequences bracketed by the real-time quantitative PCR primers (Table 1) and used precisely quantified template preparations to establish standard curves of amplification (see Methods). Quantitation of cyclophilin mRNA provided an internal standard, and data are presented as specific mRNA copy numbers/10⁶ copies of cyclophilin. As shown in FIG. 2A, Northern blot analyses clearly demonstrated higher expression of VEGF mRNA in hVEGF-High SK-MEL-2 melanoma cells in comparison with hVEGF-Low SK-MEL-2 melanoma cells. Densitometric analyses suggested that the difference was approximately 9-fold. Absolute quantitation of VEGF copy numbers with real-time quantitative RT-PCR showed that hVEGF-High transfectants expressed 5.2-fold more VEGF mRNA than the hVEGF-Low transfectants (1.5×10⁵ copies of VEGF/10⁶ copies of cyclophilin, vs.2.9×10⁴ copies of VEGF/10⁶ copies of cyclophilin) (FIG. 2B). When implanted subdermally with Matrigel in nude mice, the hVEGF-High transfectants clearly induced a significantly greater degree of skin angiogenesis than the hVEGF-Low transfectants, whereas Matrigel alone did not provoke any detectable angiogenesis (FIG. 2C). Consequently, this Matrigel hVEGF-transfectant model served as a well-controlled source of neovascularized tissue for identification of mRNAs that correlate with VEGF expression and angiogenesis.

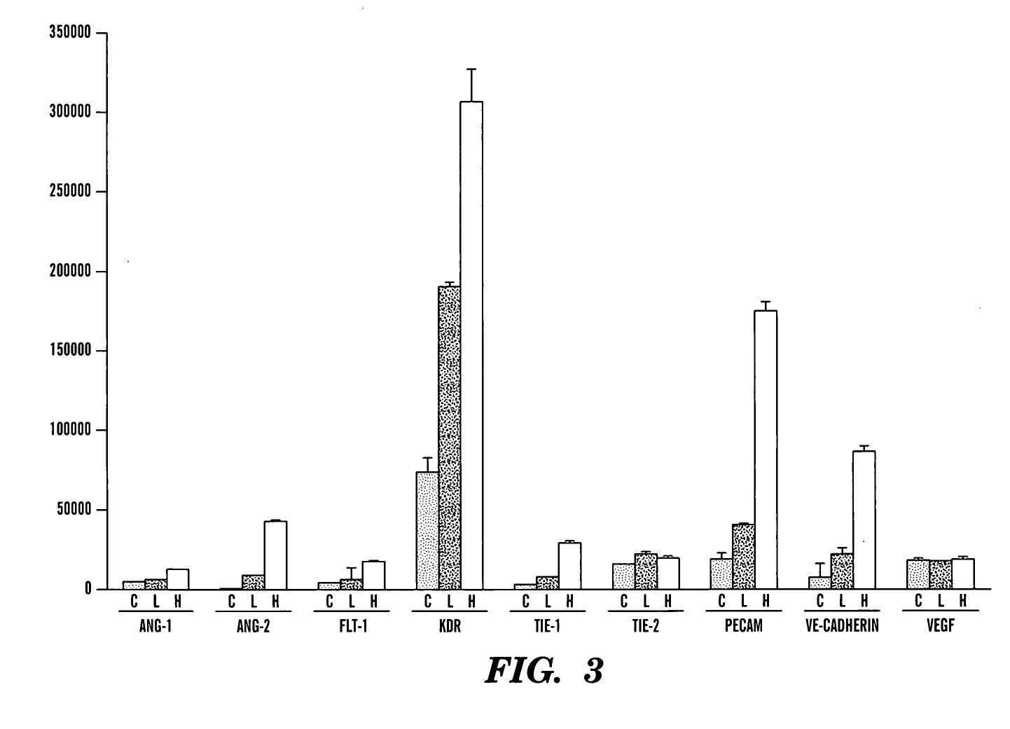

In order to quantify the candidate angiogenesis marker mRNAs expressed in the area of hVEGF-induced skin neovascularization, the implanted Matrigel was removed and cDNAs were prepared from skin specimens associated with control Matrigel, hVEGF-Low transfectants, and hVEGF-High transfectants. As shown in FIG. 3, cDNAs prepared from skin specimens associated with both hVEGF-Low and hVEGF-High transfectants clearly demonstrated higher copy numbers of Flt-1, KDR, VE-Cadherin, PECAM, Ang-1, Ang-2, and Tie-1 in comparison with skin specimens associated with Matrigel alone. Furthermore, the levels of each of these markers were highest in samples associated with the hVEGF-High transfectants. In skin specimens associated with control Matrigel, KDR had the highest mRNA copy number (7.5×10⁴ copies of KDR/10⁶ copies of cyclophilin), whereas PECAM, and Tie-2 had approximately 1.5-1.9×10⁴ copies/10⁶ copies of cyclophilin. Ang-1, Ang-2, Flt-1, Tie-1 and VE-Cadherin had the lowest copy numbers (less than 10⁴ copies/10⁶ copies of cyclophilin). In skin specimens associated with hVEGF-High transfectants, for every 10⁶ copies of cyclophilin mRNA we found approximately 3.1×10⁵ copies of KDR, 1.8×10⁵ copies of PECAM, 8.8×10⁴ copies of VE-Cadherin, 4.3¹⁰⁴ copies of Ang-2, 2.9×10⁴ copies of Tie-1, 2×10⁴ copies of Tie-2, 1.8×10⁴ copies of Flt-1, and 1.3×10⁴ copies of Ang-1. This indicates a˜35-fold induction of Ang-2, 10-fold induction of PECAM and VE-Cadherin, 8-fold induction Tie-1, and a 4-fold induction of Flt-1 and KDR in skin specimens associated with VEGF-High transfectants in comparison with control Matrigel (Table 2). Interestingly, as observed with the TRAMP tumors (FIG. 1), Tie-2 expression did not correlate with VEGF expression or the other candidate markers. All three types of skin samples (control, hVEGF-High and hVEGF-Low) showed similar levels of endogeneous VEGF expression (approximately 1.9×10⁴ copies VEGF/10⁶ copies of cyclophilin); thus the skin angiogenesis induced in human VEGF transfectants was attributable to exogenous human VEGF released from Matrigel.

TABLE 2
Fold Induction of gene expression by
VEGF-High and VEGF-Low transfectant

| Gene | Control | VEGF-Low | VEGF-High |
|---|---|---|---|
| Ang-1 | 1 | 1.29 | 2.28 |
| Ang-2 | 1 | 7.49 | 35.06 |
| Flt-1 | 1 | 1.46 | 3.64 |
| KDR | 1 | 2.56 | 4.10 |
| Tie-1 | 1 | 2.44 | 8.61 |
| Tie-2 | 1 | 1.42 | 1.25 |
| PECAM | 1 | 2.18 | 9.14 |
| VE-Cadherin | 1 | 2.76 | 10.44 |
| VEGF | 1 | 0.98 | 1.03 |

Discussion

An important early event during the progression of many cancers is the acquisition and maintenance of new blood vessels, which is now recognized both as a promising prognostic indicator and a target for therapy.^{1,20,31} In this study, we used real time RT-PCR to analyze relative changes and absolute mRNA copy numbers of a panel of potential angiogenesis markers in mouse transgenic model of prostate adenocarcinoma (TRAMP) and hVEGF-driven skin angiogenesis, respectively. Results showed an excellent quantitative correlation between VEGF and Ang-1, Ang-2, Flt-1, KDR, VE-Cadherin, PECAM, and Tie-1 in these two experimental models of angiogenesis.

The strength of a real-time quantitative RT-PCR assay lies in its potential to rapidly and precisely measure a large number of transcripts with limited material.⁴⁻⁶ By contrast, quantification with Northern blot analysis requires approximately 5000 times more RNA. Furthermore, with a precisely quantified cDNA template standard that covers the region of real-time quantitative RT-PCR primer, real-time quantitative RT-PCR is also able to measure absolute transcript copy numbers.^{7,8,32} Thus, with its superior sensitivity and accuracy, wide linear dynamic range, good intra-assay and inter-assay reproducibility, real-time quantitative RT-PCR offers potential utility in clinical diagnosis and offers an alternative route to current immunohistochemical methods for assessing angiogenesis.

Studies have shown a direct relationship between microvessel density and the level of VEGF expression during tumor progression.³²⁻³⁴ As demonstrated here, increasing the number of VEGF mRNA transcripts from 2.9×10⁴ copies to 1.5×10⁵ copies for every 10⁶ copies of cyclophilin in human SK-MEL-2 melanoma transfectants induced significantly greater degree of neovascularization in mouse skin. In particular, the panel of angiogenesis markers identified here clearly distinguished skin neovascularization induced by hVEGF-High transfectants from that induced by hVEGF-Low transfectants. This implies that proteins necessary to induce and retain blood vessels are coordinately regulated along with VEGF during tumor growth. Thus, detection of mRNA copy numbers with real-time quantitative RT-PCR is able to produce precise transcript profiles, and these profiles may serve to monitor different phases of tumor progression and efficacy of therapy.

Ang-1 is a secreted growth factor that binds to and activates the Tie-2 receptor tyrosine kinase, whereas Ang-2 binds the same receptor but fails to activate it, thus acting as a natural inhibitor of Ang-1. ^{14,21,22} Recent studies showed that Ang-2 destabilizes capillary integrity and facilitates vessel sprouting when VEGF levels are high, but causes vessel regression when VEGF levels are low.^{21,22} Our real-time quantitative RT-PCR showed that hVEGF-High transfectant contained -4.3×10⁴ copies of Ang-2 for every million copies of cyclophilin while control contained only 1200 copies, resulting in a 35-fold induction of Ang-2 expression. Thus, in agreement with others' findings, our studies suggest that higher rates of skin neovascularization are associated with higher levels of VEGF and Ang-2 expression. Interestingly, induction of Tie-2 was not correspondingly increased with VEGF in either the TRAMP or skin angiogenesis models. As determined by measurement of absolute mRNA copy number, both control and VEGF-stimulated skin samples expressed moderate yet equal levels of Tie-2 mRNA (for every million copies of cyclophilin, control skin contained 1.5×10⁴ copies of Tie-2 and vascularized skin associated with hVEGF-High transfectants expressed 2.2×10⁴ copies). Thus the ratio of Tie-2 and its ligand Ang-2 increased from 1.0/0.08 in control skin to 1.0/2.8 in hVEGF-High transfectant skin. This suggests that although Tie-2 mRNA level did not increase significantly during skin neovascularization, the pre-existing levels of Tie-2 are able to accommodate Ang-2 ligand induced signaling. This particular observation underscores the significance of rigorously quantifying mRNA copy number and illustrates that such quantification may provide important insights.

In summary, this study demonstrates a method for rigorous quantitative profiling of specific mRNAs closely correlated with VEGF expression and angiogenesis. We have defined a strategy that precisely quantifies a complex panel of marker transcripts, and our findings provide evidence that detection of absolute mRNA transcript numbers with real-time quantitative RT-PCR can serve to sensitively monitor angiogenesis. Because there may exist significant differences in gene expression among different tissues and during various phases of tumor progression, the panel of markers we have identified more accurately predicts the level of angiogenesis than singular markers and may provide for tumor and stage specific fingerprinting of neovascularization.

### REFERENCES

1. Folkman J. What is the evidence that tumors are angiogenesis dependent? J Natl Cancer Inst 1990, 82:4-6.

2. Folkman J, D'Amore P A. Blood vessel formation: what is its molecular basis? Cell 1996, 87:1153-1155.

3. Stetler-Stevenson W G. The role of matrix metalloproteinases in tumor invasion, metastasis, and angiogenesis. Surg Oncol Clin N Am 2001, 10:383-392, x.

4. Heid C A, Stevens J, Livak K J, Williams P M. Real time quantitative PCR. Genome Res 1996, 6:986-994.

5. Jung R, Soondrum K, Neumaier M. Quantitative PCR. Clin Chem Lab Med 2000, 38:833-836.

6. Freeman W M, Walker S J, Vrana K E. Quantitative RT-PCR: pitfalls and potential. Biotechniques 1999,26:112-125.

7. Aleem E A, Flohr T, Hunziker A, Mayer D, Bannasch P, Thielmann H W. Detection and quantification of protein phosphatase inhibitor-1 gene expression in total rat liver and isolated hepatocytes. Mol Cell Biochem 2001, 217:1-12.

8. Van Trappen P O, Gyselman V G, Lowe D G, et al. Molecular quantification and mapping of lymph-node micrometastases in cervical cancer. Lancet 2001, 357:15-20.

9. Breier G, Breviario F, Caveda L, et al. Molecular cloning and expression of murine vascular endothelial-cadherin in early stage development of cardiovascular system. Blood 1996, 87:630-641.

10. Caveda L, Martin-Padura I, Navarro P, et al. Inhibition of cultured cell growth by vascular endothelial cadherin (cadherin-5/VE-cadherin). J Clin Invest 1996, 98:886-893.

11. Sheibani N, Frazier W A. Thrombospondin-1, PECAM-1, and regulation of angiogenesis. Histol Histopathol 1999, 14:285-294.

12. Newman P J. The biology of PECAM-1. J Clin Invest 1997, 100:S25-29.

13. Veikkola T, Karkkainen M, Claesson-Welsh L, Alitalo K. Regulation of angiogenesis via vascular endothelial growth factor receptors. Cancer Res 2000, 60:203-212.

14. Woolf A S, Yuan H T. Angiopoietin growth factors and Tie receptor tyrosine kinases in renal vascular development. Pediatr Nephrol 2001, 16:177-184.

15. Mustonen T, Alitalo K. Endothelial receptor tyrosine kinases involved in angiogenesis. J Cell Biol 1995, 129:895-898.

16. Jones N, Dumont D J. Tek/Tie2 signaling: new and old partners. Cancer Metastasis Rev 2000, 19:13-17.

17. Partanen J, Dumont D J. Functions of Tie1 and Tie2 receptor tyrosine kinases in vascular development. Curr Top Microbiol Immunol 1999, 237:159-172.

18. Jones N. Iljin K, Dumont D J, Alitalo K. Tie receptors: new modulators of angiogenic and lymphangiogenic responses. Nat Rev Mol Cell Biol 2001, 2:257-267.

19. Ferrara N. Role of vascular endothelial growth factor in regulation of physiological angiogenesis. Am J Physiol Cell Physiol 2001, 280:C1358-1366.

20. Senger D R, Van de Water L, Brown L F, et al. Vascular permeability factor (VPF, VEGF) in tumor biology. Cancer Metastasis Rev 1993, 12:303-324.

21. Yancopoulos G D, Davis S, Gale N W, Rudge J S, Wiegand S J, Holash J. Vascular-specific growth factors and blood vessel formation. Nature 2000, 407:242-248.

22. Holash J, Wiegand S J, Yancopoulos G D. New model of tumor angiogenesis: dynamic balance between vessel regression and growth mediated by angiopoietins and VEGF. Oncogene 1999, 18:5356-5362.

23. Asahara T, Chen D, Takahashi T, et al. Tie2 receptor ligands, angiopoietin-1 and angiopoietin-2, modulate VEGF-induced postnatal neovascularization. Circ Res 1998, 83:233-240.

24. Papapetropoulos A, Garcia-Cardena G, Dengler T J, Maisonpierre P C, Yancopoulos G D, Sessa W C. Direct actions of angiopoietin-1 on human endothelium: evidence for network stabilization, cell survival, and interaction with other angiogenic growth factors. Lab Invest 1999, 79:213-223.

25. Foster B A, Kaplan P J, Greenberg N M. Peptide growth factors and prostate cancer: new models, new opportunities. Cancer Metastasis Rev 1998, 17:317-324.

26. Huss W J, Maddison L A, Greenberg N M. Autochthonous mouse models for prostate cancer, past, present and future. Semin Cancer Biol 2001, 11:245-260.

27. Gingrich J R, Barrios R J, Morton R A, et al. Metastatic prostate cancer in a transgenic mouse. Cancer Res 1996, 56:4096-4102.

28. Shih S C, Claffey K P. Regulation of human vascular endothelial growth factor mRNA stability in hypoxia by heterogeneous nuclear ribonucleoprotein L. J Biol Chem 1999, 274:1359-1365.

29. Robinson G S, Ju M, Shih S C, et al. Nonvascular role for VEGF: VEGFR-1, 2 activity is critical for neural retinal development. Faseb J 2001, 15:1215-1217.

30. Trandinh C C, Pao G M, Saier M H, Jr. Structural and evolutionary relationships among the immunophilins: two ubiquitous families of peptidyl-prolyl cis-trans isomerases. Faseb J 1992, 6:3410-3420.

31. Folkman J, Ingber D. Inhibition of angiogenesis. Semin Cancer Biol 1992, 3:89-96.

32. Straume O, Akslen L A. Expression of vascular endothelial growth factor, its receptors (FLT-1, KDR) and TSP-1 related to microvessel density and patient outcome in vertical growth phase melanomas. Am J Pathol 2001, 159:223-235.

33. Toi M, Inada K, Suzuki H, Tominaga T. Tumor angiogenesis in breast cancer: its importance as a prognostic indicator and the association with vascular endothelial growth factor expression. Breast Cancer Res Treat 1995, 36:193-204.

34. Seo Y, Baba H, Fukada T, Takashima M, Sugimachi K. High expression of vascular endothelial growth factor is associated with liver metastasis and a poor prognosis for patients with ductal pancreatic adenocarcinoma. Cancer 2000, 88:2239-2245.

The references cited herein and throughout the specification are herein incorporated by reference in their entirety.

1. A method of determining an absolute copy number of an expressed gene of interest in a biological specimen comprising the steps of:
a) preparing at least one first standard set comprising a serial dilution of at least two different dilutions with known copy numbers of a first vector in a buffer, wherein the vector comprises a vector backbone and a sufficient portion of the coding region of a housekeeping gene;
b) preparing at least one second standard set comprising a serial dilution of at least two different dilutions with known copy numbers of a second vector in a buffer, wherein the vector comprises a vector backbone and a sufficient portion of the coding region of a gene of interest:
c) performing a PCR reaction on the first standard set with housekeeping primers capable of amplifying the housekeeping gene in the first vector and measuring the amount of PCR product at a predetermined time point of the PCR reaction using each dilution of the step a) and plotting the amount of amplified PCR product at the time point against the known copy number of the first vector in said dilution thereby creating a first standard curve;
d) performing a PCR reaction on the second standard set with gene of interest primers capable of amplifying the gene of interest in the second vector and measuring the amount of PCR product at a predetermined time point of the PCR reaction using each dilution of the step b) and plotting the amount of amplified PCR product at the time point against the known copy number of the second vector thereby creating a second standard curve;
e) obtaining a biological specimen;
f) producing a cDNA sample from mRNAs contained in the biological specimen;
g) obtaining a housekeeping gene copy number by performing a PCR reaction on the sample of step f) with the housekeeping primers, measuring the amount of the PCR product and using the amount to determine the copy number of the housekeeping gene on the first standard curve;
h) obtaining a gene of interest copy number by performing a PCR reaction on the sample of step f) with the gene of interest primers, measuring the amount of the PCR product and using the amount to determine the copy number of the gene of interest on the second standard curve; and
i) determining the absolute copy number of the gene of interest by determining the ratio of copy numbers of step h) over step g).

2-5. (canceled)

6. The method of claim 1, wherein the housekeeping gene is cyclophilin.

7. The method of claim 1, or 6, wherein the gene of interest is selected from the group consisting of Ang-1, Ang-2, Fit-1, KDR, Tie-1, Tie-2, PECAM, VE-Cadherin, and VEGF, and any combination thereof.

8. The method of claim 7, wherein the gene of interest primers are SEQ ID NO: 1, SEQ ID NO: 2, SEQ ID NO: 4-SEQ ID NO: 12, and SEQ ID NO: 14-SEQ ID NO: 20 and the housekeeping primers are SEQ ID NO: 3 and SEQ ID NO: 13.

9. The method of claims 1 and 3, wherein the measuring of the amount of the amplification product is performed using a real-time PCR.
